(19) United States
(12) Patent Application Publication
(10) Pub. No.: US 2011/0034802 A1
(43) Pub. Date: Feb. 10, 2011

(54) SYSTEMS, METHODS, AND APPARATUS FOR IMAGING AN IMPLANTABLE DEVICE AND METHODS FOR MANUFACTURING

(75) Inventors: Sanjay Shrivastava, Irvine, CA (US); Alexander Nikanorov, Palo Alto, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 12/536,389

(22) Filed: Aug. 5, 2009

(51) Int. Cl. A61F 2/01; A61B 8/00; A61B 17/00; A61F 2/06; B23P 11/00; C21D 7/06; B23K 26/00; B21D 22/00; B21C 23/00

(52) U.S. Cl. 600/424; 606/200; 623/1.34; 606/213; 29/428; 72/53; 219/121.85; 72/352; 72/253.1

(57) ABSTRACT

A method for imaging an implantable device is described. The method may include positioning an implantable device within a patient. Ultrasonic energy may be transmitted toward the implantable device. The returning ultrasonic energy may be analyzed to determine a first characteristic of the implantable device. A method for manufacturing an implantable device is also described. The method may include selecting a first size dimension for a plurality of ultrasonically visible portions. The method may further include selecting a first spacing dimension for the plurality of ultrasonically visible portions. An implantable device may be formed. The plurality of ultrasonically visible portions may be formed into the first outer surface. An implantable device is also described. The implantable device may include a first outer surface. The first outer surface may include a first ultrasonically visible portion having a first size dimension.

### CROSS-REFERENCE TO RELATED APPLICATIONS

This application hereby incorporates U.S. Provisional Patent Application No. 61/014,395, filed Dec. 17, 2007, and entitled “Methods for Imaging a Delivery System” by reference in its entirety.

### FIELD OF THE INVENTION

The present invention relates generally to medical devices. More particularly the present invention relates to systems, methods, and apparatus for imaging an implantable device and methods for manufacturing.

### BACKGROUND OF THE INVENTION

Catheterization and interventional procedures, such as stenting or placement of a lumen filter, generally are performed by inserting a hollow needle through a patient's skin and tissue into the vascular system. A guide wire may be advanced through the needle and into the patient's blood vessel accessed by the needle. The needle is then removed, enabling an introducer sheath to be advanced over the guide wire into the vessel, e.g., in conjunction with or subsequent to a dilator.

A catheter or other device may then be advanced through a lumen of the introducer sheath and over the guide wire into a position for performing a medical procedure (for example, inserting a stent or lumen filter into a body lumen). Thus, the introducer sheath may facilitate introducing various devices into the vessel, while minimizing trauma to the vessel wall and/or minimizing blood loss during a procedure. Because implantable devices are typically implanted in a patient, it may be desirable to image the implantable device during and/or after implantation.

Accordingly, systems, methods, and apparatus for imaging an implantable device and methods for manufacturing may also be useful.

### BRIEF SUMMARY

A method for imaging an implantable device is described. The method may include positioning an implantable device within a patient. The implantable device may include a first outer surface and a plurality of ultrasonically visible portions formed into the first outer surface. At least one of the plurality of ultrasonically visible portions may have a first size dimension. At least two of the plurality of ultrasonically visible portions may be separated by a first spacing dimension. Ultrasonic energy may be transmitted toward the implantable device. The returning ultrasonic energy may be analyzed to determine a first characteristic of the implantable device.

In some embodiments, analyzing the returning ultrasonic energy to determine a first characteristic of the implantable device includes determining the relative position of the implantable device within the patient. In further embodiments, determining the relative position of the implantable device within the patient includes determining the relative position of the implantable device within the patient during positioning of the implantable device within the patient.

An attempt may be made, in some embodiments, to deploy the implantable device within the patient. In further embodiments, determining the relative position of the implantable device within the patient includes determining the relative position of the implantable device within the patient prior to deployment of the implantable device within the patient.

In some embodiments, it may be determined whether the implantable device was deployed. In further embodiments, if the implantable device did not deploy, the relative position of the implantable device within the delivery apparatus may be determined.

The first characteristic may include the orientation of the implantable device, the location of the implantable device within the patient, the state of the implantable device within the patient, or other characteristics, or combinations thereof. In further embodiments, the state of the implantable device within the patient includes whether the implantable device is in a deployed state.

In some embodiments, the first outer surface may be a part of an ultrasonically visible marker operatively associated with the implantable device. The first outer surface, in further embodiments, may be integral to the implantable device. In still further embodiments, the plurality of ultrasonically visible portions may cover the first outer surface of the implantable device. The implantable device, in some embodiments, may be a vascular filter, a closure element, a valve frame, a stent, other implantable devices, or combinations thereof.

In another embodiment, a method for manufacturing an implantable device is described. The method includes selecting a first size dimension for a plurality of ultrasonically visible portions. A first spacing dimension may be selected for the plurality of ultrasonically visible portions. The first spacing dimension may be used to determine the spacing between at least two of the plurality of ultrasonically visible portions. An implantable device may be formed having a first outer surface. The plurality of ultrasonically visible portions may be formed into the first outer surface using the selected first size dimension and the selected first spacing dimension.

In some embodiments, forming a plurality of ultrasonically visible portions into the first outer surface may be performed by plastically deforming at least a portion of the first outer surface. In further embodiments, forming a plurality of ultrasonically visible portions into the first outer surface may be performed by removing at least a portion of the first outer surface. Forming a plurality of ultrasonically visible portions into the first outer surface, in still further embodiments, may be performed using a shot peening process, a shot blasting process, a laser texturing process, a textured die, an extrusion die, other forming processes, or combinations thereof.

The first size dimension, in some embodiments, may be selected to create an approximate surface area of one of the plurality of ultrasonically visible portions. The approximate surface area of one of the plurality of ultrasonically visible portions, in further embodiments, may be predetermined to facilitate ultrasonic visibility.

In some embodiments, the ultrasonically visible portions may have at least one surface that is non-parallel to a portion of the first outer surface proximate the ultrasonically visible portions. In further embodiments, the first size dimension may be selected to create an approximate depth of one of the plurality of ultrasonically visible portions. The approximate depth of one of the plurality of ultrasonically visible portions may be predetermined, in still further embodiments, to facilitate ultrasonic visibility. In yet further embodiments, the first outer surface may be a part of an ultrasonically visible marker operatively associated with the implantable device.

An embodiment of an implantable device is described. The implantable device may include a first outer surface that may include a first ultrasonically visible portion having a first size dimension. The implantable device may include a second ultrasonically visible portion having a first size dimension. The second ultrasonically visible portion may be separated from said first ultrasonically visible portion by a spacing dimension.

In some embodiments, the first ultrasonically visible portion and/or the second ultrasonically visible portion may be formed by removing portions of said first surface, by plastically deforming portions of said first surface, by other processes, or combinations thereof. The first size dimension of the first ultrasonically visible portion and/or the first size dimension of the second ultrasonically visible portion, in further embodiments, may be about the same dimension. In still further embodiments, the first ultrasonically visible portion may include a second size dimension. The second size dimension may include a width.

Other aspects and features of the present invention will become apparent from consideration of the following description in conjunction with the accompanying drawings.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 illustrates an embodiment of a surface of an implantable device.

FIG. 2 illustrates another embodiment of a surface of an implantable device.

FIG. 3 illustrates a further embodiment of a surface of an implantable device.

FIG. 4 illustrates a still further embodiment of a cutaway surface of an implantable device.

FIG. 5 illustrates an embodiment of an implantable device.

FIG. 6 illustrates another embodiment of an implantable device.

FIG. 7 illustrates a further embodiment of an implantable device.

FIG. 8 illustrates an embodiment of a method for imaging an implantable device.

FIG. 9 illustrates another embodiment of a method for imaging an implantable device.

FIG. 10 illustrates an embodiment of a method for manufacturing an embodiment of an implantable device.

It should be noted that the figures are not drawn to scale and that elements of similar structures or functions are generally represented by like reference numerals for illustrative purposes throughout the figures. It also should be noted that the figures are only intended to facilitate the description of embodiments of the present invention.

### DETAILED DESCRIPTION

The embodiments described herein extend generally to methods, systems, and apparatus for imaging an implantable device and methods for manufacturing. It may be desirable for implantable devices to be visible under ultrasonic signals. In addition, the use of ultrasonic imaging devices often allows for noninvasive imaging. Implantable devices are typically implanted under fluoroscopy. However, fluoroscopes are relatively expensive. Thus, it is generally impractical for most rooms in a hospital, clinic, office, or other medical facility to have a fluoroscope. Ultrasonic imaging devices, on the other hand, are often found in many parts of a medical facility. For example, an intensive care unit will typically have an ultrasonic imaging device while generally not having a fluoroscope. Furthermore ultrasonic imaging devices may be desirable in some body locations, such as a heart valve.

In many cases, implantable device are not readily visible in ultrasonic images. To facilitate the imaging of an implantable device, it may be desirable to alter a surface of the implantable device. For example, the surface may be roughened in an organized way. Examples of processes for roughing the surface of the implantable device may include laser texturing, cold drawing with a textured roll, cold extrusion through textured dies, shot peening, shot blasting, fabrication using the powder processing route, other processes, or combinations thereof.

Processes such as shot blasting and/or shot peening may be used with particles of known or unknown sizes and/or shapes. The particle sizes and/or shapes may be determined to increase the visibility of the surface during ultrasonic imaging.

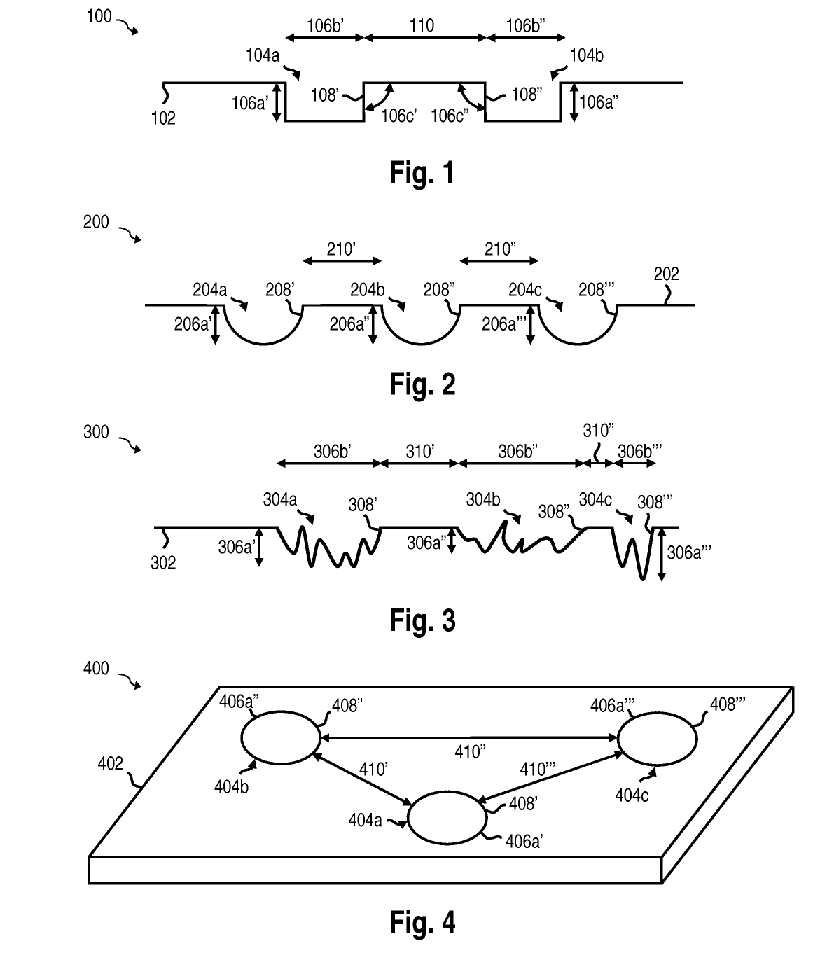

FIG. 1 illustrates an embodiment of a surface of an implantable device 100. Implantable device may include endoprostheses, drug delivery stents, drug delivery catheters, stent-grafts, grafts, drug delivery balloons, guidewires, orthopedic implants, PFO closure devices, pacemaker leads, dental implants, fixation screws, indwelling catheters, implantable filters, ocular implants, pharmacotherapeutic implants, blood-contacting components of extracorporeal devices, staples, filters, needles, tubes, coils, wires, clips, screws, sensors, plates, conduits, portions thereof, combinations thereof, and/or other implantable devices.

The implantable device 100 may include an outer surface 102 that may include a first ultrasonically visible portion 104a and/or a second ultrasonically visible portion 104b. The first and/or second ultrasonically visible portions 104a, 104b may be concave with respect to the rest of the outer surface 102. For example, the first and/or second ultrasonically visible portions 104a, 104b may have a generally polygonal shape. Alternatively, the first and/or second ultrasonically visible portions 104a, 104b may have other shapes. For example, the first and/or second ultrasonically visible portions 104a, 104b may be part of a pyramid, cone, sphere, other shape, may have a generally random geometric pattern, or combinations thereof.

In the present embodiment, the first and/or second ultrasonically visible portions 104a, 104b may be formed by removing material from the outer surface 102 of the implantable device 100. In other embodiments, the first and/or second ultrasonically visible portions 104a, 104b may be formed by plastically deforming the outer surface 102 of the implantable device 100.

The first and/or second ultrasonically visible portions 104a, 104b may include at least one size dimension 106a′, 106a″, 106b′, 106b″, 106c′, 106c″. Size dimensions may include a depth, a maximum depth, an average depth, a width, an angle, a length, a radius, a diameter, a diagonal length, a surface area, and/or other size dimensions.

In the embodiment of FIG. 1, the first size dimension 106a′, 106a″, the second size dimension 106b′, 106b″, and/or the third size dimension 106c′, 106c″ of the first and/or second ultrasonically visible portions 104a, 104b may be a depth from an inner surface 108′, 108″ of the first and/or second ultrasonically visible portion 104a, 104b to the outer surface 102, a width of the first and/or second ultrasonically visible portion 104a, 104b, and/or an angle from the outer surface 102 to the inner surface 108′, 108″ of the first and/or second ultrasonically visible portion 104a, 104b, respectively.

The depth may range from about 1 μm to about 100 μm. The width may range from about 1 μm to about 100 μm. The length (not shown) may range from about 1 μm to about 100 μm.

In the embodiment shown in FIG. 1, the size dimensions 106a″, 106b″, 106c″ of the second ultrasonically visible portion 104b may be approximately the same as the size dimensions 106a′, 106b′, 106c′ of the first ultrasonically visible portion 104b. In other embodiments, the size dimensions 106a″, 106b″, 106c″ of the second ultrasonically visible portion 104b may be different from the size dimensions 106a′, 106b′, 106c′ of the first ultrasonically visible portion 104b. In further embodiments, some of the size dimensions 106a″, 106b″, 106c″ of the second ultrasonically visible portion 104b may be different from the size dimensions 106a′, 106b′, 106c′ of the first ultrasonically visible portion 104b and some of the size dimensions 106a″, 106b″, 106c″ of the second ultrasonically visible portion 104b may be approximately the same as the size dimensions 106a′, 106b′, 106c′ of the first ultrasonically visible portion 104b.

As described above, the third size dimension 106c′, 106c″ of the first and/or second ultrasonically visible portions 104a, 104b, in the present embodiment, may include an angle between the outer surface 102 and an inner surface 108′, 108″ of the first and/or second ultrasonically visible portions 104a, 104b. In embodiments, where the angle may be approximately more or less than one hundred and eighty degrees (180), the implantable device 100 may deflect ultrasonic signals (i.e. waves) making the implantable device 100 visible to an ultrasonic imaging device. Other size dimensions may facilitate visibility of the implantable device 100 to an ultrasonic imaging device. For example, the depth, width, length, and/or other size dimensions of an ultrasonically viewable portion may facilitate visibility.

The first and second ultrasonically visible portions 104a, 104b may be separated by a spacing dimension 110. The spacing dimension 110 may be selected to facilitate the ultrasonic visibility of the implantable device 100. For example, ultrasonically visible portions may be separated by a spacing dimension 110 ranging from about 1 μm to about 100 μm.

FIG. 2 illustrates another embodiment of a surface of an implantable device 202. The elements of the implantable device 202 of this embodiment may be functionally similar to the elements of the implantable device 102 previously described above and shown in FIG. 1 in most respects, wherein certain features will not be described in relation to this embodiment wherein those components may function in the manner as described above and are hereby incorporated into this alternative embodiment described below. Like structures and/or components are given like reference numerals.

The implantable device 200 may include an outer surface 202. The outer surface 202 may include a first ultrasonically visible portion 204a, a second ultrasonically visible portion 204b, and/or a third ultrasonically visible portion 204c. The first, second, and/or third ultrasonically visible portions 204a, 204b, 204c may be concave with respect to the rest of the outer surface 202. For example, the first ultrasonically visible portion 204a may have a generally elliptical shape.

The first, second, and/or third ultrasonically visible portions 204a, 204b, 204c, in the present embodiment, may all have approximately the same shape. In other embodiments, the first, second, and/or third ultrasonically visible portions 204a, 204b, 204c may all have different shapes or may have some shapes that are different and some that are approximately the same. The first, second, and/or third ultrasonically visible portions 204a, 204b, 204c, in the present embodiment, have an approximately spherical shape. In other embodiments, the first, second, and/or third ultrasonically visible portions 204a, 204b, 204c may have other shapes and/or a combination of shapes.

In the present embodiment, first, second, and/or third ultrasonically visible portions 204a, 204b, 204c may be formed by removing material from the outer surface 202 of the implantable device 200. In other embodiments, the first, second, and/or third ultrasonically visible portions 204a, 204b, 204c may be formed by plastically deforming the outer surface 202 of the implantable device 200. In further embodiments, the first, second, and/or third ultrasonically visible portions 204a, 204b, 204c may be formed by a combination of removing material from or plastically deforming the outer surface 202 of the implantable device 200.

The first, second, and/or third ultrasonically visible portions 204a, 204b, 204c may include at least one size dimension 206a′, 206a″, 206a′″. In the present embodiment, the first size dimension 206a′, 206a″, 206a′″ may include a radius. The radius may range from about 1 μm to about 100 μm.

In the embodiment shown in FIG. 2, the size dimensions 206a′, 206a″, 206a′″ of the first, second, and/or third ultrasonically visible portions 204a, 204b, 204c may be approximately the same. In other embodiments, the size dimensions may be different from each other. In further embodiments, some of the size dimensions may be different from each other and some of the size dimensions may be approximately the same.

As described above, first, second, and/or third ultrasonically visible portions 204a, 204b, 204c may have an elliptical shape. The first, second, and/or third ultrasonically visible portions 204a, 204b, 204c may include an inner surface 208′, 208″, 208′″ that may be generally non-parallel with the outer surface 202. Non-parallel inner surfaces may deflect ultrasonic signals (i.e. waves) making the implantable device 200 visible to an ultrasonic imaging device. Other inner surfaces may facilitate visibility of the implantable device 200 to an ultrasonic imaging device. In addition, an inner surface that is parallel to the outer surface 202 may deflect ultrasonic signals (i.e. waves) to make the implantable device 200 visible to an ultrasonic imaging device. For example, the inner surfaces 110′, 110″ shown in FIG. 1 may be offset (i.e. via a depth size dimension) to facilitate the ultrasonic visibility of the implantable device 100.

The first, second, and/or third ultrasonically visible portions 204a, 204b, 204c may be separated by a spacing dimension 210′, 210″. The spacing dimensions 210′, 210″ may be selected to facilitate the ultrasonic visibility of the implantable device 200. For example, ultrasonically visible portions may be separated by a spacing dimension ranging from about 1 μm to about 100 μm.

In the embodiment shown in FIG. 2, the spacing dimensions 210′, 210″ may be approximately the same. In other embodiments, the spacing dimensions 210′, 210″ may be different from each other. In further embodiments, some of the spacing dimensions may be different from each other and some of the spacing dimensions may be approximately the same.

FIG. 3 illustrates a further embodiment of a surface of an implantable device 300. The elements of the implantable device 300 of this embodiment may be functionally similar to the elements of the implantable devices 100, 200 previously described above and shown in FIGS. 1 and 2 in most respects, wherein certain features will not be described in relation to this embodiment wherein those components may function in the manner as described above and are hereby incorporated into this alternative embodiment described below. Like structures and/or components are given like reference numerals.

The implantable device 300 may include an outer surface 302. The outer surface 302 may include a first ultrasonically visible portion 304a, a second ultrasonically visible portion 304b, and/or a third ultrasonically visible portion 304c. The first, second, and/or third ultrasonically visible portions 304a, 304b, 304c may include at least one inner surface 308′, 308″, 308′″. In the present embodiment, the first, second, and/or third ultrasonically visible portions 304a, 304b, 304c may be generally concave with respect to the rest of the outer surface 302. For example, portions of the inner surface 308″ of the second ultrasonically visible portion 304b may be convex with respect to the outer surface 302, however, the second ultrasonically visible portion 304b may, nonetheless, be generally concave as a majority of the inner surface 308″ of the second ultrasonically visible portion 304b is also concave. In other embodiments, the first, second, and/or third ultrasonically visible portions 304a, 304b, 304c may be generally convex with respect to the rest of the outer surface 302. This may be accomplished by, for example, heating portions of the outer surface 302 to expand the first, second, and/or third ultrasonically visible portions 304a, 304b, 304c beyond the outer surface 302.

The first, second, and/or third ultrasonically visible portions 304a, 304b, 304c may be separated by a spacing dimension 310′, 310″. The spacing dimensions 310′, 310″ may be selected to facilitate the ultrasonic visibility of the implantable device 300. For example, ultrasonically visible portions may be separated by a spacing dimension 310′, 310″ ranging from about 1 μm to about 100 μm. In the embodiment shown in FIG. 3, the spacing dimensions 310′, 310″ may vary. In other embodiments, the spacing dimensions 310′, 310″ may be approximately the same. In further embodiments, some of the spacing dimensions may be different from each other and some of the spacing dimensions may be approximately the same.

The first, second, and/or third ultrasonically visible portions 304a, 304b, 304c, in the present embodiment, may all have varying shapes. In other embodiments, the first, second, and/or third ultrasonically visible portions 304a, 304b, 304c may all have the same shape or may have some shapes that are different and some that are approximately the same. The first, second, and/or third ultrasonically visible portions 304a, 304b, 304c, in the present embodiment, have a generally convex random shape, such as, for example, a shape formed by a shot peening or shot blasting process.

In the present embodiment, the first, second, and/or third ultrasonically visible portions 304a, 304b, 304c may be formed by plastically deforming portions of the outer surface 302 of the implantable device 300. In other embodiments, the first, second, and/or third ultrasonically visible portions 304a, 304b, 304c may be formed by removing material from the outer surface 302 of the implantable device 300. In further embodiments, the first, second, and/or third ultrasonically visible portions 304a, 304b, 304c may be formed by a combination of removing material from or plastically deforming the outer surface 302 of the implantable device 300.

The first, second, and/or third ultrasonically visible portions 304a, 304b, 304c may include at least one size dimension 306a′, 306a″, 306a′″, 306b′, 306b″, 306b′″. Size dimensions may include a depth, a width, an angle, a length, a radius, a diameter, a diagonal length, a surface area, and/or other size dimensions.

In the embodiment of FIG. 3, the first size dimension 306a′, 306a″ , 306a′″ and/or the second size dimension 306b′, 306b″, 306b′″ of first, second, and/or third ultrasonically visible portions 304a, 304b, 304c may be an approximate maximum depth from the outer surface 302 and/or a width of the ultrasonically visible portions 304a, 304b, 304c, respectively. The maximum depth may range from about 1 μm to about 100 μm. The width may range from about 1 μm to about 100 μm. In other embodiments, the first size dimension 306a′, 306a″, 306a′″ may include an approximate average depth, an angle between the inner surface 308′, 308″ and the outer surface 302, and/or other size dimensions.

The size dimensions 306a′, 306a″, 306a′″, 306b′, 306b″, 306b′″ of first, second, and/or third ultrasonically visible portions 304a, 304b, 304c may be different from each other. In other embodiments, the size dimensions may be approximately the same. In further embodiments, some of the size dimensions may be different from each other and some of the size dimensions may be approximately the same.

FIG. 4 illustrates a still further embodiment of a cutaway surface of an implantable device 400. The elements of the implantable device 400 of this embodiment may be functionally similar to the elements of the implantable devices 100, 200, 300 previously described above and shown in FIGS. 1, 2, and 3 in most respects, wherein certain features will not be described in relation to this embodiment wherein those components may function in the manner as described above and are hereby incorporated into this alternative embodiment described below Like structures and/or components are given like reference numerals.

The implantable device 400 may include an outer surface 402. The outer surface 402 may include a first ultrasonically visible portion 404a, a second ultrasonically visible portion 404b, and/or a third ultrasonically visible portion 404c. The first, second, and/or third ultrasonically visible portions 404a, 404b, 404c may be concave with respect to the rest of the outer surface 402. For example, the first ultrasonically visible portion 404a may have a generally elliptical shape.

The first, second, and/or third ultrasonically visible portions 404a, 404b, 404c, in the present embodiment, may all have approximately the same shape. In other embodiments, the first, second, and/or third ultrasonically visible portions 404a, 404b, 404c may all have different shapes or may have some shapes that are different and some that are approximately the same.

In the present embodiment, first, second, and/or third ultrasonically visible portions 404a, 404b, 404c may be formed by removing material from the outer surface 402 of the implantable device 400. In other embodiments, the first, second, and/or third ultrasonically visible portions 404a, 404b, 404c may be formed by plastically deforming the outer surface 402 of the implantable device 400. In further embodiments, the first, second, and/or third ultrasonically visible portions 404a, 404b, 404c may be formed by a combination of removing material from or plastically deforming the outer surface 402 of the implantable device 400.

The first, second, and/or third ultrasonically visible portions 404a, 404b, 404c may include at least one size dimension 406a′, 406a″, 406a′″. In the present embodiment, the first size dimension 406a′, 406a″, 406a′″ may include an approximate surface area. The surface area may range from about 1 μm to about 100 μm.

In the embodiment shown in FIG. 4, the size dimensions 406a′, 406a″, 406a′″ of the first, second, and/or third ultrasonically visible portions 404a, 404b, 404c may be approximately the same. In other embodiments, the size dimensions may be different from each other. In further embodiments, some of the size dimensions may be different from each other and some of the size dimensions may be approximately the same.

As described above, first, second, and/or third ultrasonically visible portions 404a, 404b, 404c may have an elliptical shape. The first, second, and/or third ultrasonically visible portions 404a, 404b, 404c may include an inner surface 408′, 408″, 408′″ that may be generally non-parallel with the outer surface 402.

The first, second, and/or third ultrasonically visible portions 404a, 404b, 404c may be separated by a spacing dimension 410′, 410″, 410′″. The spacing dimensions 410′, 410″, 410′″ may be selected to facilitate the ultrasonic visibility of the implantable device 400. For example, ultrasonically visible portions may be separated by a spacing dimension 410′, 410″, 410′″ ranging from about 1 μm to about 100 μm. In the embodiment shown in FIG. 4, the spacing dimensions 410′, 410″, 410′″ may be different from each other. In other embodiments, the spacing dimensions 410′, 410″, 410′″ may be approximately the same. In further embodiments, some of the spacing dimensions may be different from each other and some of the spacing dimensions may be approximately the same.

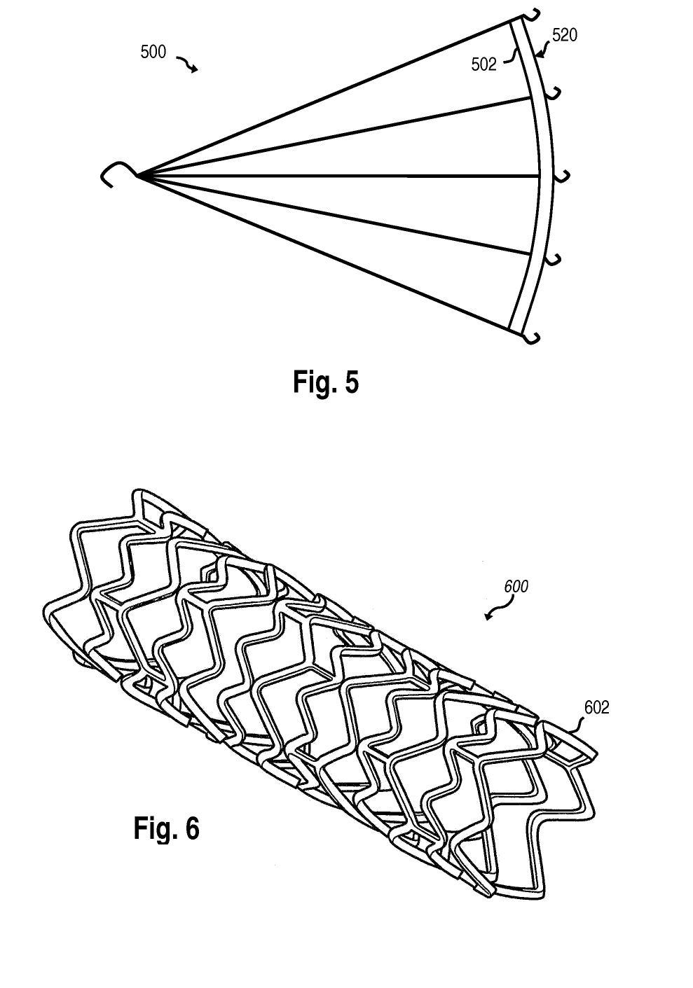

FIG. 5 illustrates an embodiment of an implantable device 500. The implantable device 500 of the present embodiment may be a lumen filter, such as a vena cava filter. The lumen filter may include a structure configured and dimensioned as a filter to be used within a lumen of an animal. In the embodiment shown in FIG. 5, the implantable device 500 may include an ultrasonically visible marker 520. The ultrasonically visible marker 520 may be operatively associated with the implantable device 520. For example, the ultrasonically visible marker 520 may be a processed tissue or polymer leaflet that may be attached to the implantable device 500. The ultrasonically visible marker 520 may include an outer surface 502 that may include a plurality of ultrasonically visible portions (such as the ultrasonically visible portions 104, 204, 304, 404 previously described above and shown in FIGS. 1-4). In other embodiments, the ultrasonically visible portions may be integral to portions of or the entire implantable device 500.

FIG. 6 illustrates another embodiment of an implantable device 600. The implantable device 600 of the present embodiment may be a stent. The stent may include a structure configured and dimensioned as a stent to be used within a lumen of an animal. In the embodiment shown in FIG. 6, the implantable device 600 may include an outer surface 602, portions of which (or the entire outer surface) may include a plurality of ultrasonically visible portions (such as the ultrasonically visible portions 104, 204, 304, 404 previously described above and shown in FIGS. 1-4). In other embodiments, the implantable device 600 may include an ultrasonically visible marker (such as the ultrasonically visible marker 520 previously described above and shown in FIG. 5).

The outer surface 602 may include more than the extreme outer surface. In some embodiments, the outer surface 602 may include an ablumenal surface of the implantable device 600. In further embodiments, the outer surface 602 may include a side surface of the implantable device 600.

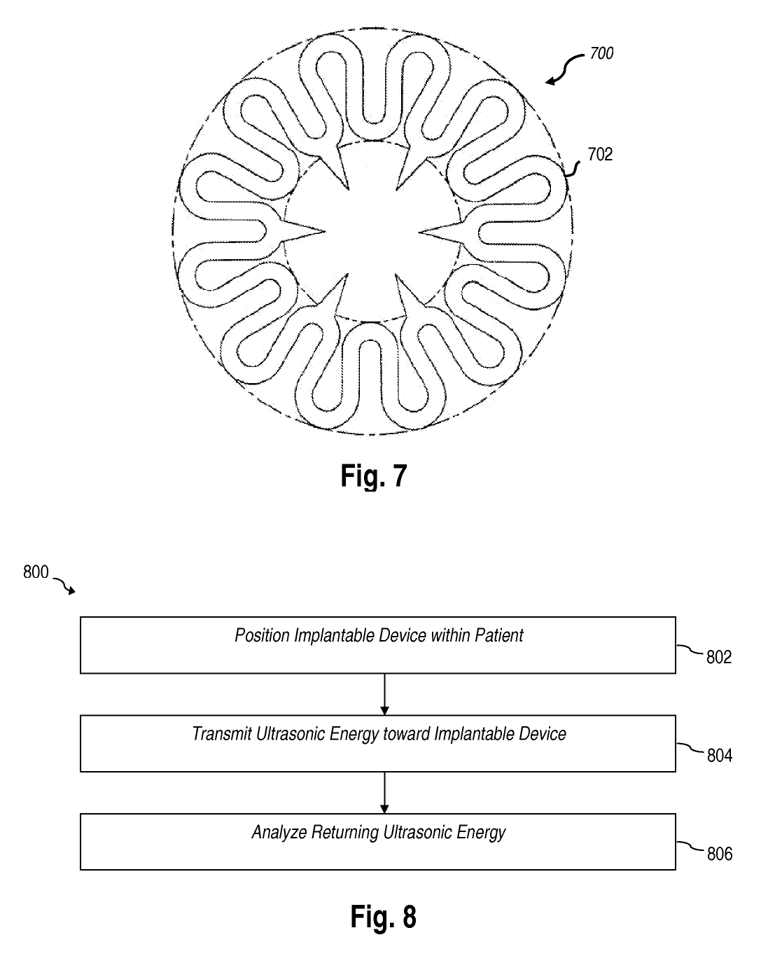

FIG. 7 illustrates a further embodiment of an implantable device 700. The implantable device 700 of the present embodiment may be an engaging element. The engaging element may include a structure configured and dimensioned as an engaging element to be used to engage tissue. In the embodiment shown in FIG. 7, the implantable device 700 may include an outer surface 702, portions of which (or the entire outer surface) may include a plurality of ultrasonically visible portions (such as the ultrasonically visible portions 104, 204, 304, 404 previously described above and shown in FIGS. 1-4). In other embodiments, the implantable device 700 may include an ultrasonically visible marker (such as the ultrasonically visible marker 520 previously described above and shown in FIG. 5).

FIG. 8 illustrates an embodiment of a method 800 for imaging an implantable device. In the present embodiment, the method 800 may be used in conjunction with the implantable devices 100, 200, 300, 400, 500, 600, 700 and components described in connection with FIGS. 1-7, and/or any other systems and/or apparatus for imaging an implantable device described herein.

An implantable device may be positioned within a patient, as represented by block 802. Positioning an implantable device within a patient may include positioning the implantable device in a desired location and/or in a desired orientation. For instance, a lumen filter may be positioned in a desired location within a body lumen, such as within the inferior vena cava. In another example, a valve frame may be positioned in a desired location, such as the aortic valve.

Ultrasonic energy may be transmitted toward the implantable device, as represented by block 804. The ultrasonic energy may be transmitted toward the implantable device via an ultrasonic imaging device. A technician may transmit ultrasonic energy into a patient before, during, and/or after positioning the implantable device within the patient.

The returning ultrasonic energy may be analyzed, as represented by block 806. Analyzing the returning ultrasonic energy may include producing an image, for example, on a monitor. In another example, a microprocessor and/or other data processing device may analyze the returning ultrasonic energy. Analyzing the returning ultrasonic energy may further include producing some other indicator, such as sound.

Analyzing the returning ultrasonic energy may include determining a first characteristic of the implantable device. The characteristics of the implantable device may include the relative position of the implantable device within the patient, the orientation of the implantable device within the patient, the location of the implantable device within the patient, the state of the implantable device within the patient (i.e. whether the implantable device is in a deployed state), and/or other characteristics or combinations thereof. Analyzing the returning ultrasonic energy may be performed before, during, and/or after positioning the implantable device within the patient.

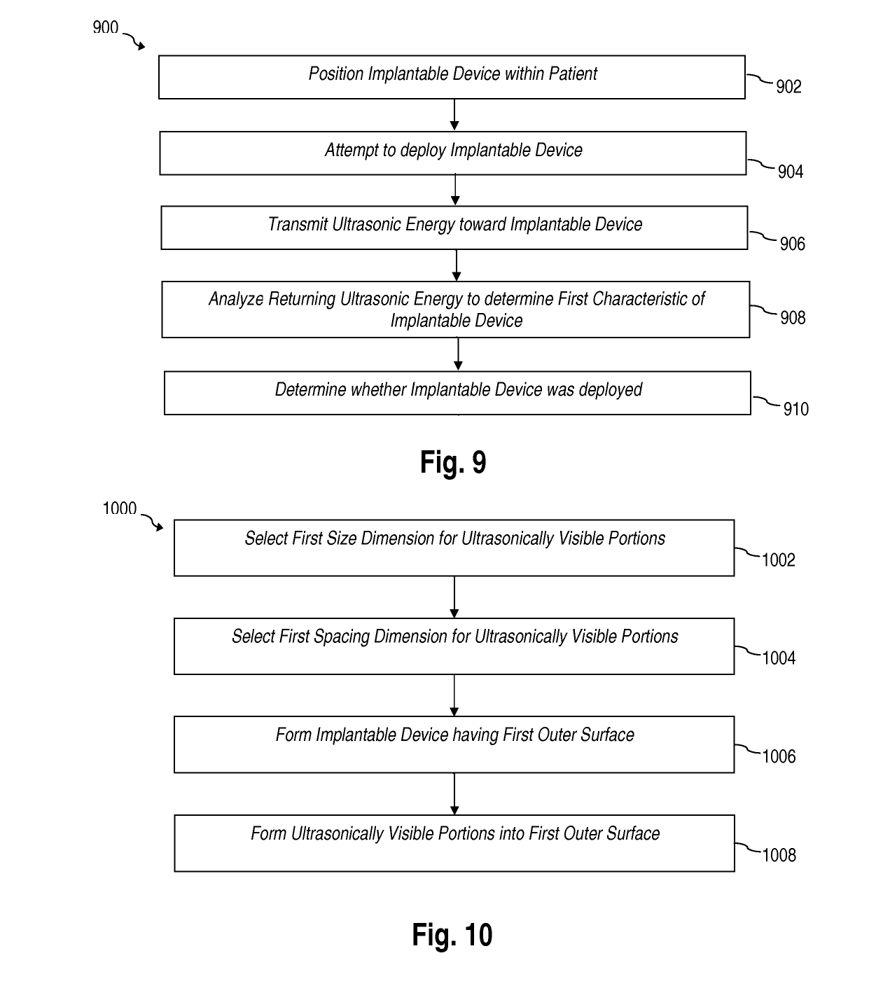

FIG. 9 illustrates another embodiment of a method 900 for imaging an implantable device. In the present embodiment, the method 900 may be used in conjunction with the implantable devices 100, 200, 300, 400, 500, 600, 700 and components described in connection with FIGS. 1-7, and/or any other systems and/or apparatus for imaging an implantable device described herein.

The method 900 of this other embodiment may be functionally similar to that of the method 800 previously described above and shown in FIG. 8 in most respects, wherein certain features will not be described in relation to this other embodiment wherein those method components may be performed in the manner as described above and are hereby incorporated into this alternative embodiment described below.

An implantable device may be positioned within a patient, as represented by block 902. An attempt to deploy the implantable device may be made, as represented by block 904. Deploying an implantable device may include, for example, transitioning an implantable device from a collapsed to an expanded state (or vice versa), anchoring a lumen filter within a body lumen, engaging the inner surface of a body lumen with the outer surface of a stent, engaging tissue with a tissue engaging portion of an engaging element, and/or other types of deployments.

Ultrasonic energy may be transmitted toward the implantable device, as represented by block 906. The returning ultrasonic energy may be analyzed to determine a first characteristic of the implantable device, as represented by block 908. It may be determined whether the implantable device was deployed, as represented by block 910. Determining whether the implantable device was deployed may include determining whether the implantable device successfully transitioned from a collapsed to an expanded state (or vice versa), whether a lumen filter successfully anchored within a body lumen, whether the outer surface of a stent successfully engaged the inner surface of a body lumen, whether a tissue engaging portion of an engaging element successfully engaged tissue, and/or other indicators of successful deployments.

FIG. 10 illustrates an embodiment of a method 1000 for manufacturing an embodiment of an implantable device. In the present embodiment, the method 1000 may be used in conjunction with the implantable devices 100, 200, 300, 400, 500, 600, 700 and components described in connection with FIGS. 1-7, and/or any other systems and/or apparatus for imaging an implantable device described herein.

The method 1000 may include selecting a first size dimension for at least one ultrasonically visible portion, as represented by block 1002. The first size dimension may be similar to the size dimensions described above. For example, the first size dimension may include a depth (i.e. a maximum or average depth), a width, an angle, a length, a radius, a diameter, a diagonal length, a surface area, and/or other size dimensions. Additional size dimensions may also be selected, such as a second dimension. Selecting a size dimension may include selecting a die, roller, shot for a shot peening or shot blasting procedure, and/or other forming device to form the at least one ultrasonically visible portion.

A first spacing dimension for the at least one ultrasonically visible portion may be selected, as represented by block 1004. The first spacing dimension may be similar to the spacing dimensions described above. Selecting a spacing dimension may include selecting a die, roller, shot for a shot peening or shot blasting procedure, and/or other forming device to form the at least one ultrasonically visible portions.

In the present embodiment, both a first size dimension and a first spacing dimension for the at least one ultrasonically visible portion may be selected. In other embodiments, only a first size dimension or a first spacing dimension for the at least one ultrasonically visible portion may be selected.

The first size dimension may be selected to create an approximate surface area, an approximate depth, and/or other dimension of one of the at least one ultrasonically visible portion. The approximate surface area, approximate depth, and/or other dimension of one of the at least one ultrasonically visible portion may be predetermined to facilitate ultrasonic visibility. The first size dimension may be selected to create at least one surface of at least one ultrasonically visible portion that is non-parallel to a portion of the first outer surface proximate the ultrasonically visible portion.

An implantable device may be formed, as represented by block 1006. The implantable device may include a first outer surface, as described above. Forming the implantable device may include forming tissue engaging portions (i.e. in the case of an engaging element implantable device and/or lumen filter implantable device), support members (i.e. in the case of a stent implantable device, lumen filter implantable device, and/or closure element implantable device), and/or other components of the implantable device. The implantable device may be formed from a base material. The base material may be a shape memory material, such as alloys of nickel-titanium, and/or other materials.

Ultrasonically visible portions may be formed into the first outer surface of the implantable device, as represented by block 1008. The ultrasonically visible portions may be formed by removing material from the outer surface of the implantable device, by plastically deforming the outer surface of the implantable device, and/or other forming processes. The ultrasonically visible portions may be formed using a shot peening process, a laser texturing process, a textured die, an extrusion die, and/or other processes.

The ultrasonically visible portions may be formed into the first outer surface of the implantable device using the selected first size dimension, the selected first spacing dimension, and/or other selected dimensions. Additional size, spacing, other dimensions, and/or combinations of the same may be selected and/or used to form the ultrasonically visible portions.

The invention is susceptible to various modifications and alternative means, and specific examples thereof have been shown by way of example in the drawings and are herein described in detail. It should be understood, however, that the invention is not to be limited to the particular devices or methods disclosed, but to the contrary, the invention is to cover all modifications, equivalents, and alternatives falling within the spirit and scope of the claims.

We claim:

1. A method for imaging an implantable device, the method comprising:
positioning an implantable device within a patient, the implantable device including:
a first outer surface; and
a plurality of ultrasonically visible portions formed into the first outer surface, at least one of the plurality of ultrasonically visible portions having a first size dimension, at least two of the plurality of ultrasonically visible portions being separated by a first spacing dimension;
transmitting ultrasonic energy toward the implantable device; and
analyzing the returning ultrasonic energy to determine a first characteristic of the implantable device.

2. The method of claim 1, wherein analyzing the returning ultrasonic energy to determine a first characteristic of the implantable device further comprises determining the relative position of the implantable device within the patient.

3. The method of claim 2, wherein determining the relative position of the implantable device within the patient further comprises determining the relative position of the implantable device within the patient during positioning of the implantable device within the patient.

4. The method of claim 2, further comprising attempting to deploy the implantable device within the patient, wherein determining the relative position of the implantable device within the patient further comprises determining the relative position of the implantable device within the patient prior to deployment of the implantable device within the patient.

5. The method of claim 4, further comprising determining whether the implantable device was deployed.

6. The method of claim 4, further comprising determining the relative position of the implantable device within the delivery apparatus if the implantable device did not deploy.

7. The method of claim 1, wherein the first characteristic includes the orientation of the implantable device.

8. The method of claim 1, wherein the first characteristic includes the location of the implantable device within the patient.

9. The method of claim 1, wherein the first characteristic includes the state of the implantable device within the patient.

10. The method of claim 9, wherein the state of the implantable device within the patient includes whether the implantable device is in a deployed state.

11. The method of claim 1, wherein the first outer surface is a part of an ultrasonically visible marker operatively associated with the implantable device.

12. The method of claim 1, wherein the first outer surface is integral to the implantable device.

13. The method of claim 1, wherein the plurality of ultrasonically visible portions cover the first outer surface of the implantable device.

14. The method of claim 1, wherein the implantable device is a vascular filter.

15. The method of claim 1, wherein the implantable device is a closure element.

16. The method of claim 1, wherein the implantable device is a valve frame.

17. The method of claim 1, wherein the implantable device is a stent.

18. A method for manufacturing an implantable device, the method comprising:
selecting a first size dimension for a plurality of ultrasonically visible portions;
selecting a first spacing dimension for the plurality of ultrasonically visible portions, the first spacing dimension being used to determine the spacing between at least two of the plurality of ultrasonically visible portions;
forming an implantable device having a first outer surface; and
forming the plurality of ultrasonically visible portions into the first outer surface using the selected first size dimension and the selected first spacing dimension.

19. The method of claim 18, wherein forming a plurality of ultrasonically visible portions into the first outer surface is performed by plastically deforming at least a portion of the first outer surface.

20. The method of claim 18, wherein forming a plurality of ultrasonically visible portions into the first outer surface is performed by removing at least a portion of the first outer surface.

21. The method of claim 18, wherein forming a plurality of ultrasonically visible portions into the first outer surface is performed using a shot peening or shot blasting process.

22. The method of claim 18, wherein the plurality of ultrasonically visible portion is formed using a laser texturing process.

23. The method of claim 18, wherein the plurality of ultrasonically visible portion is formed using a textured die.

24. The method of claim 18, wherein the plurality of ultrasonically visible portion is formed using an extrusion die.

25. The method of claim 18, wherein the first size dimension is selected to create an approximate surface area of one of the plurality of ultrasonically visible portions.

26. The method of claim 25, wherein the approximate surface area of one of the plurality of ultrasonically visible portions is predetermined to facilitate ultrasonic visibility.

27. The method of claim 18, wherein the ultrasonically visible portions have at least one surface that is non-parallel to a portion of the first outer surface proximate the ultrasonically visible portions.

28. The method of claim 18, wherein the first size dimension is selected to create an approximate depth of one of the plurality of ultrasonically visible portions.

29. The method of claim 28, wherein the approximate depth of one of the plurality of ultrasonically visible portions is predetermined to facilitate ultrasonic visibility.

30. The method of claim 18, wherein the first outer surface is a part of an ultrasonically visible marker operatively associated with the implantable device.

31. An implantable device, comprising:
a first outer surface including:
a first ultrasonically visible portion having a first size dimension; and
a second ultrasonically visible portion having a first size dimension, said second ultrasonically visible portion being separated from said first ultrasonically visible portion by a spacing dimension.

32. The implantable device of claim 31, wherein said first ultrasonically visible portion and said second ultrasonically visible portion being formed by removing portions of said first surface.

33. The implantable device of claim 31, wherein said first ultrasonically visible portion and said second ultrasonically visible portion being formed by plastically deforming portions of said first surface.

34. The implantable device of claim 31, wherein said first size dimension of said first ultrasonically visible portion and said first size dimension of said second ultrasonically visible portion are about the same dimension.

35. The implantable device of claim 31, wherein said first ultrasonically visible portion further includes a second size dimension.

36. The implantable device of claim 31, wherein said second size dimension includes a width.
